(19) United States
(12) Patent Application Publication
(10) Pub. No.: US 2003/0211484 A1
(43) Pub. Date: Nov. 13, 2003

(54) Sequence lineage evaluation interface

(76) Inventor: Keith Ball, Berkeley, CA (US)

Correspondence Address: LARGESCALE BIOLOGY CORPORATION, BANK OF AMERICA PLAZA, SUITE 2020, 414 UNION STREET, NASHVILLE, TN, 37219, US

(21) Appl. No.: 10/144,430

(22) Filed: May 13, 2002

(51) Int. Cl. C12Q 1/68; G06F 19/00; G01N 33/48; G01N 33/50

(52) U.S. Cl. 435/6; 702/20

(57) ABSTRACT

A computer implemented interface provides a graphical representation of a plurality of either nucleic or amino acid sequences that have segments in common, the graphical representation enabling a user to visually compare and evaluate sequence data. Specifically, the graphical interface displays sequence data sets of either nucleic acid or amino acid sequence data, the sequence data sets including at least a first parent sequence and at least one daughter sequence, where the daughter sequence potentially includes sequence segments inherited from the first parent sequence and/or from a second parent sequence. Each daughter sequence is compared to the parent sequences, and common segments or sequence portions are displayed with a color or grayscale code to making it easy for a user to identify segments common to both parent and daughter sequences, as well as identify those segments that are not common to parent and daughter sequence.

### FIELD OF THE INVENTION

The invention relates to an interface for reviewing nucleic and amino acid sequences. The invention relates further to an interface that provides a graphical representation of a plurality of either nucleic and/or amino acid sequences that have segments in common, the graphical representation enabling a user to visually compare and evaluate sequence data.

### BACKGROUND OF THE INVENTION

In recent years evaluation of nucleic acid sequence data has become an ever increasing part of many aspects of modern biological sciences. Previously, scientists evaluated sinusoidal curves outputted from a sequencer by observing peaks in the curves to determine sequences of tested amino acids and gene sequences. Such evaluations were time consuming and tedious. Advances in software evaluation of such curves has advanced to the point where the sequencers themselves are provided with programming that evaluates such curves and determines the identified sequence. Such sequencers output the graphically represented curves along with the identified sequence. However, it is time consuming and difficult to put separate sequences side by side and evaluate the sequences in pairs or in groups of sequences. Hence, there is a need for more sophisticated tools for comparing and evaluating pairs or groups of sequences.

Gene manipulation and protein manipulation have advanced tremendously in recent years. Scientists are able to cleave, anneal and extend genes to yield new and unique sequences. For instance, in U.S. patent application Ser. No. 09/775,049, filed Jan. 31, 2001, entitled METHODS FOR HOMOLOGY-DRIVEN REASSEMBLY OF NUCLEIC ACID SEQUENCES, parent gene sequences are cleaved, and annealed to produce daughter sequences that include portions of both parent genes. The daughter sequences may include mismatched segment portions that are subsequently repaired to eliminate the mismatched segment portions. Once repaired, it is desirable to compare the daughter sequences with the parent sequences. Since a plurality of daughters is typically produced, it is necessary to compare all the daughters with the parents. However, since hundreds of daughters are produced, the task of comparing the final daughter sequences with the parent sequences is a daunting task. Hence there is a need for an efficient and effective means for comparing the parent/daughter sequences.

Gene sequences and proteins are studied for any of a variety of reasons, for instance in genetic testing, forensic examinations, and research purposes. Libraries of mutant sequences can be generated by any of a variety of methods known in the art such as chemical or physical mutagenesis, mutagenic PCR, oligonucleotide-directed mutagenesis, or growth in a DNA repair deficient microorganism (mutator strain). In addition, related sequences can be found in gene families within an organism, or in homologous genes from related organisms. In any application where gene sequences must be compared, there is a need for a reliable, efficient and effective means for inspecting and comparing a plurality of sequences. Hence, there is a need for a new way of comparing and evaluating gene sequences and amino acid sequences.

### SUMMARY OF THE INVENTION

The invention relates to computer software that compares parent sequences with daughter sequences to identify inheritance patterns. The invention further relates to a graphical interface that displays both parent and daughter sequences side by side enabling a user to compare the sequences and make meaningful interpretations of the compared sequences.

### BRIEF DESCRIPTION OF THE DRAWINGS

FIG. 1 is a representation of one example of sequence manipulation, showing parent sequences and subsequently produced daughter sequences;

FIG. 2 is a block diagram showing an example of a computer system configured to receive and evaluate sequence data;

FIG. 3 is a flowchart showing basic steps in a process that compares sets of sequence data and displays the evaluated sequence data on a computer display;

FIG. 4 is an example of an input interface displayed on a computer monitor, showing at least two alternative ways of inputting identified sequences;

FIG. 5 is an enlarged portion of the display depicted in FIG. 4, showing in greater detail a means for selecting color representations on a computer monitor of the subsequently displayed data;

FIG. 6 is an example of an initial overview of data where colored blocks indicate comparison information between parent genomic sequences and daughter sequences, where the color coding was determined by selections made using the interface representations depicted in FIGS. 4 and 5;

FIG. 7A is an enlarged portion of the display depicted in FIG. 6, showing in greater detail buttons linking the display to further displays, and a portion of the block color coding representing two parent sequences and a plurality of daughter sequences;

FIG. 7B is an enlarged portion of the display depicted in FIG. 6, showing in greater detail a table listing daughter sequences and related statistical analysis data;

FIG. 8 is an example of a display of two parent sequences and a selected one of the plurality of daughter sequences shown side by side, with the two parent sequences in the first two upper rows and the selected daughter shown below the two parent sequences;

FIG. 9 is an enlarged portion of the display depicted in FIG. 8 showing in greater detail a portion of the display of the two parent sequences and the selected daughter sequence;

FIG. 10 is an example of a display of data where colored blocks indicate comparison information between parent protein sequences and daughter protein sequences, where the color coding was determined by selections made using the interface representations depicted in FIGS. 4 and 5;

FIG. 11 is an enlarged portion of the display depicted in FIG. 10, showing in greater detail a portion of the block color coding representing two parent protein sequences and a plurality of daughter protein sequences;

FIG. 12 is an example of a display of two parent protein sequences and a selected one of the plurality of daughter protein sequences shown adjacent to one another, with the two parent sequences in the first two upper rows and the selected daughter shown below the two parent sequences for easy visual comparison;

FIG. 13 is an enlarged portion of the display depicted in FIG. 12 showing in greater detail a portion of the display of the two parent protein sequences and the selected daughter protein sequence; and

FIG. 14 is an enlarged portion of a display showing two parent sequences and several daughter sequences with markers indicating cross-over from a segment with homology to one parent, to another segment with homology to the other parent.

### DETAILED DESCRIPTION OF THE INVENTION

Definition of Terms:

Parent sequence: A parent sequence may be either a protein sequence, a DNA, cDNA, RNA or other nucleotide sequence that is manipulated in any of a variety of ways such that the initial sequence may possibly be altered to produce a daughter sequence.

Daughter sequence: A daughter sequence may be a nucleotide or a protein sequence produced from a set of parent sequences by manipulation or recombination of the parent sequences by any of a variety of means.

Phreds (Phred score): Phreds or Phred scores are measures of the quality of a base call for a multi-fluorescence nucleic acid electrophoresis gel. Phred uses simple Fourier methods to examine the four base traces in the region surrounding each point in the data set in order to predict a series of evenly spaced predicted locations. That is, it determines where the peaks would be centered if there were no compressions, dropouts, or other factors shifting the peaks from their “true” locations. Next phred examines each trace to find the centers of the actual, or observed, peaks and the areas of these peaks relative to their neighbors. The peaks are detected independently along each of the four traces so many peaks overlap. A dynamic programming algorithm is used to match the observed peaks detected in the second step with the predicted peak locations found in the first step. Phred evaluates the trace surrounding each called base using four or five quality value parameters to quantify the trace quality. It uses a quality value lookup table to assign the corresponding quality value. The quality value is related to the base call error probability by the formula QV=−10*log_{—}10(P_e) where P_e is the probability that the base call is an error.

The present invention relates to computer software and corresponding hardware for receiving sequence data, processing that sequence data, and displaying the data in a manner that enables a user, such as a scientist or technician, to visually evaluate the processed data and make conclusions about the processed data.

In current genetic and proteomic research it is often necessary to compare a nucleotide sequence or protein sequence with other sequences. The present invention provides a means for visually comparing a plurality of sets of sequence data and viewing that data as either nucleotide sequence data or protein sequence data.

In one embodiment of the present invention, it is possible to display parent sequences and compare them to daughter sequences where the daughter sequences were generated by manipulation or mutation of the parent sequences. In this embodiment of the present invention, a plurality of daughter sequences may be displayed side by side with one or more parent sequences so that the changes between the parent sequence or parent sequences can be easily observed. However, it should be understood that the interface of the present invention may be used to compare any combination of sequences, regardless of whether or not there is a parent/daughter relationship between the sequences.

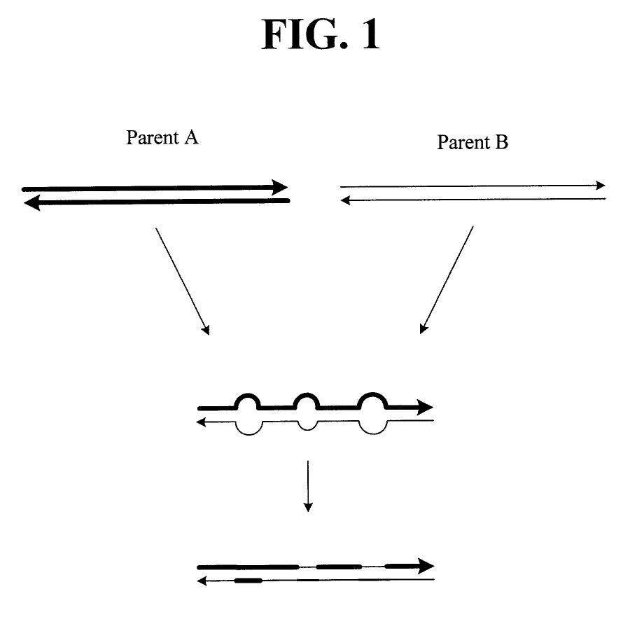

FIG. 1 shows an example of manipulated sequences. At the top of FIG. 1, a pair of parent sequences is depicted. The parent sequences are manipulated by any of a variety of means, such as the techniques set forth in co-pending U.S. patent application Ser. No. 09/775,049, filed Jan. 31, 2001, entitled METHODS FOR HOMOLOGY-DRIVEN REASSEMBLY OF NUCLEIC ACID SEQUENCES, or U.S. patent application Ser. No. 10/066,390, filed Feb. 1, 2002, entitled A METHOD OF INCREASING COMPLEMENTARITY IN A HETERODUPLEX both commonly assigned to Large Scale Biology Corporation, Vacaville, Calif. Beneath the parent sequences, the manipulated sequences are depicted showing several mismatched portions. The mismatched portions are repaired by the process set forth in co-pending U.S. patent application Ser. No. 09/775,049, filed Jan. 31, 2001, entitled METHODS FOR HOMOLOGY-DRIVEN REASSEMBLY OF NUCLEIC ACID SEQUENCES, and/or U.S. patent application Ser. No. 10/066,390, filed Feb. 1, 2002, entitled A METHOD OF INCREASING COMPLEMENTARITY IN A HETERODUPLEX, which are incorporated herein by reference in their entirety.

At the bottom of FIG. 1 the daughter sequences of the parent sequences are shown in a repaired state, where some of the repaired portions of the sequence inherit their repaired portions from parent sequence A, and some repaired portions inherit their repaired sequences from parent sequence B. The present invention evaluates each of the sequences of the parents and the daughters and determines the following:

which portions of parent sequence A are the same as portions of parent sequence B

which portions of each daughter sequence were inherited from parent A

which portions of each daughter sequence were inherited from parent B

which portions of each daughter sequence mutated (i.e., were not inherited from either parent A or parent B)

which portions of each daughter sequence differ from the consensus of the parents (i.e. a PCR error)

where alignment gaps in the sequences occur

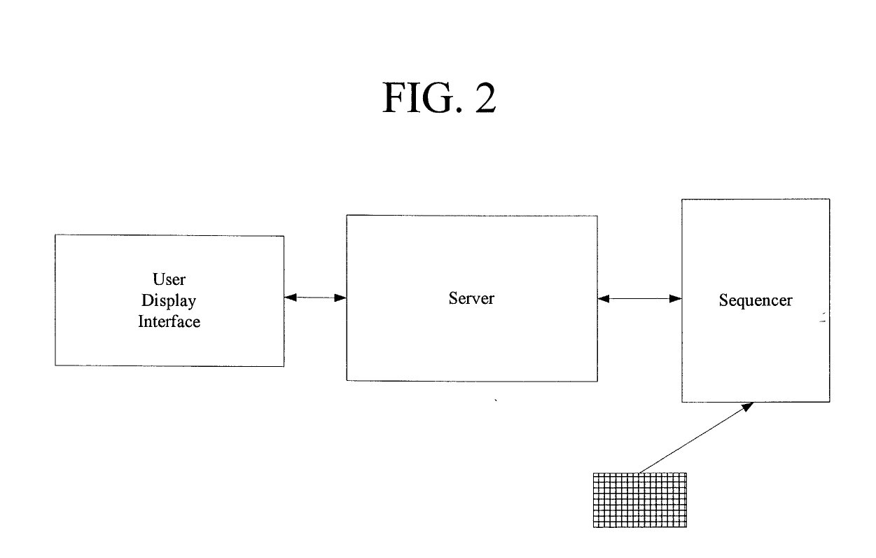

The software algorithm of the present invention may be run on a computer, such as the server depicted in FIG. 2. The server receives data from either a sequencer also depicted in FIG. 2, or from a user accessing the server from an interface, also depicted in FIG. 2. The sequencer typically determines sequences by evaluating a plurality of samples stored in, for instance, a 96-well plate, also depicted in FIG. 2. Sequence data may be accessed by the server directly from the sequences, or may be provided by the user via the interface.

The server includes typical computer related components such as memory, disk drive storage, local area network communications, etc. In the following description, storage of data is referred to repeatedly. Data storage by the server is effected by any of a variety of means. For instance, data is stored in electronic memory and data is also stored in a more permanent form in such devices as a hard disk drive, tape drive, CD-ROM or CD-Read drive, CD-RW drive, or other similar permanent storage device. However, it should be understood that in the context of the following description that data storage or storage refers to maintaining the stored data in either or both memory or permanent storage on a disk or tape based storage system.

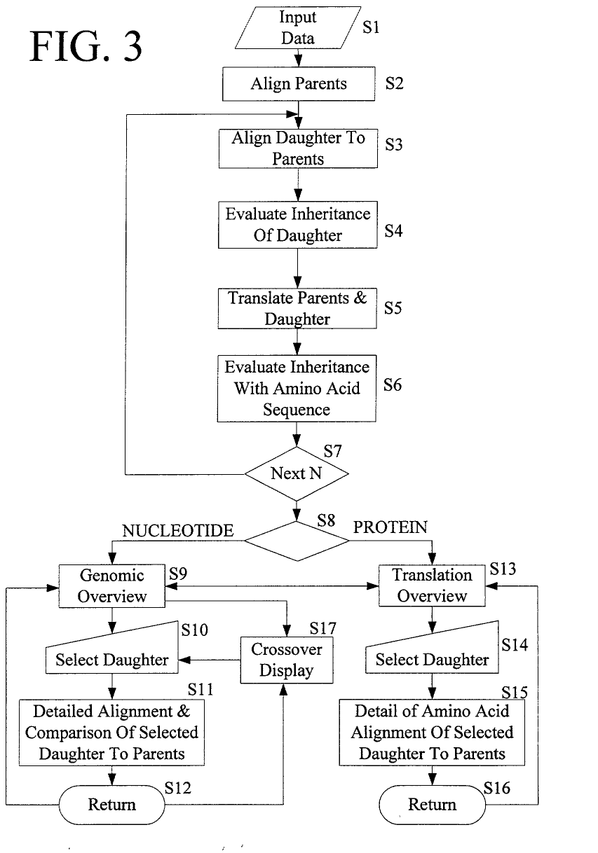

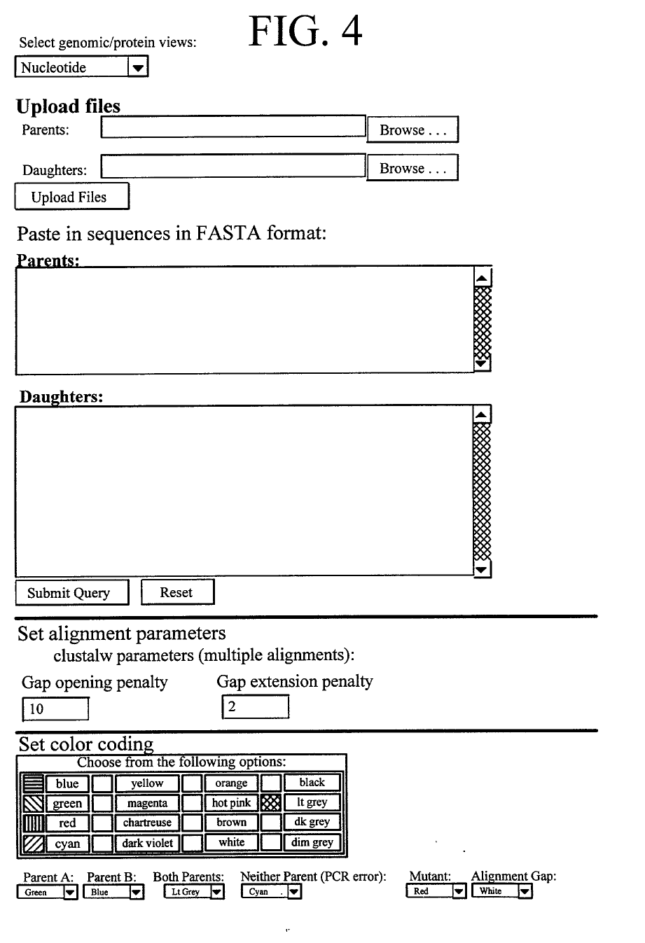

FIG. 3 is a flowchart showing details of the operations performed in accordance with the present invention. First, at step S1, sequence data is inputted, as is also depicted in FIG. 4. As depicted in FIG. 4, a plurality of inputting options is available. The user may browse for files existing in the server or user interface, using the Browse buttons shown at the upper portion of the display image captured in FIG. 4, or the sequences may be typed in the boxes depicted in the mid-portion of FIG. 4, or sequences may be cut and pasted into the boxes in the mid-portion of FIG. 4.

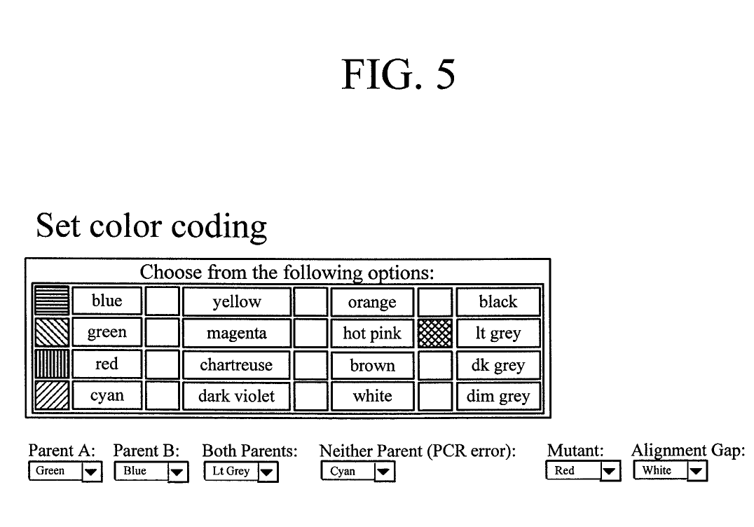

At the bottom of the display captured in FIG. 4, there is a table that indicates color selection for outputted data, as will become clearer in the description of the outputted data hereinbelow. As shown at the bottom of FIG. 4 and on an enlarged scale in FIG. 5, a series of selections are made to determine the color of the subsequently displayed output of data. For instance, a user can select the specific color or shade of grey indicating the origin of each portion of each sequence to be shown in later generated displays in the selected color or selected shade of grey. Specifically, the color of a display representing portions of each sequence that originated in either the parent sequence A or the patent sequence B can be selected. The color used in the screen display to represent those portions of each sequence common to both parents can be selected. The color of the computer screen display representing those portions of a sequence that are not found in either parent sequence can be selected. Further, the user can select the color used to display or represent mutant segments of a sequence and the color used to represent in a display any gap in the identified sequence alignment.

It should be understood that default values for the color can be predetermined and if no selection is made for the color displays, then the default colors or grey scales are automatically used.

In addition, values of parameters governing the pairwise alignment of sequences, such as gap opening and gap extension penalties, may also be selected from the interface. Appropriate default values will be used which yield a good alignment in most cases. Alternatively, a choice of pairwise alignment algorithms may be used.

Once the sequences are inputted and color selections have been made, operation moves to step S2 in FIG. 3 where the parent sequences are aligned, and their differences from one another highlighted. Any of a variety of alignment algorithms may be employed, such as CLUSTALW, a widely used alignment software program. However, it should be understood that alignment of the two parent sequences may be performed by any appropriate algorithm, not only the CLUSTALW software. Those portions of parent sequence A that are common to corresponding portions of sequence B are identified and the data stored in the server until needed for either display or further evaluation.

Next, as shown in FIG. 3, at step S3, a first daughter sequence is aligned to the parents. Specifically, those portions of the first daughter that align with the aligned portions of both parent sequences A and B are identified and the identification information stored in the server until needed for either display or further evaluation. At step S4 in FIG. 3, the current daughter sequence is evaluated to identify which portions were inherited from parent sequence A and which portions were inherited from parent sequence B. Appropriate inheritance data is stored until needed.

At step S5, the parent sequences and daughter sequence are then translated into protein sequences and the translations are stored in a data file. At step S6, the translated parent and daughter sequences are evaluated with respect to amino acid sequence inheritance, and appropriate data is stored.

At step S7 in FIG. 3, a determination is made whether or not more daughters are to be evaluated. A counter advances to the next N daughter sequence and steps S3, S4, S5 and S6 are repeated until all daughters are evaluated. Once the daughter sequences have all been evaluated, the evaluated information is displayable in several formats. At step S8, a decision is made concerning selection of data made in the screen display depicted in FIG. 4. In the upper left hand corner of the display in FIG. 4, either Nucleotide or Protein may be selected. If Nucleotide is selected, then operation moves to step S9 in FIG. 3.

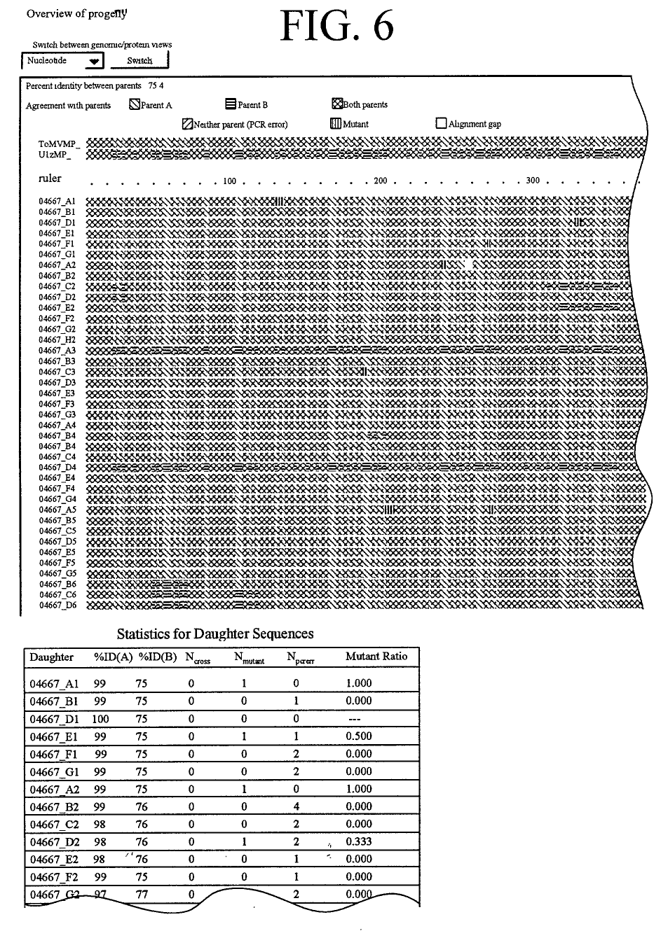

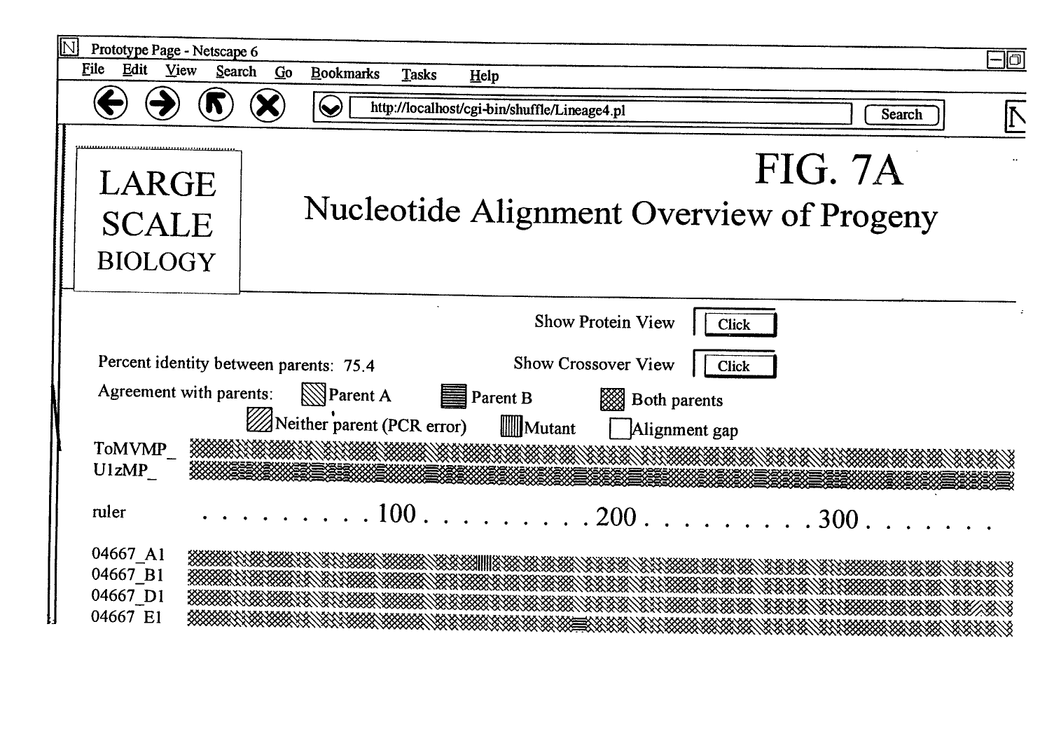

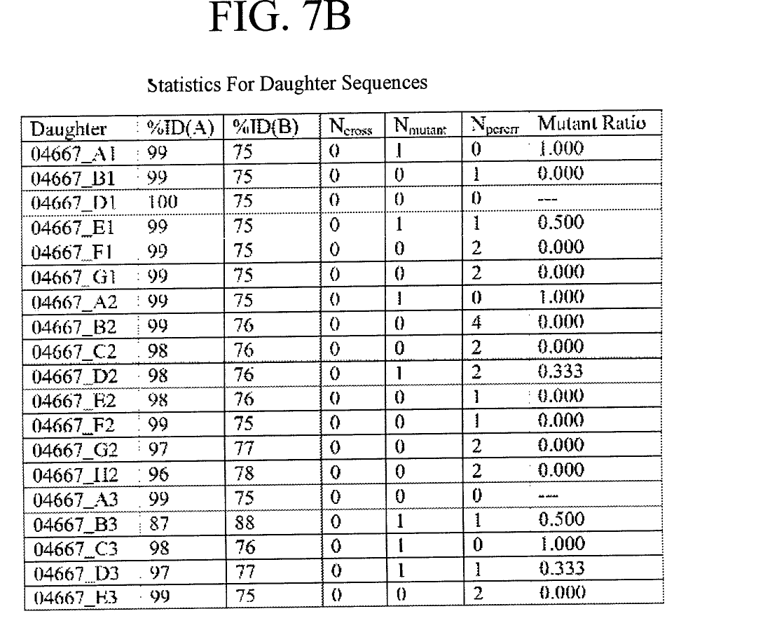

At step S9, data is collected from storage and displayed on the computer display interface, for example, as depicted in FIG. 6. FIG. 6 is a captured screen display that includes the selected color displays of a plurality of nucleotide sequences, with the parent sequences A and B shown at the top of the display, and a plurality of daughters displayed in alignment under the parent sequences. The various colors shown in each daughter row indicate the origin of that part of the sequence. Portions of the display in FIG. 6 are shown on an enlarged scale in FIGS. 7A and 7B. In FIG. 7A, the selected or default color scheme is depicted above the parent sequences A and B along with buttons that link to other displays, as is described in greater detail below. In the example depicted in FIG. 7A, parent sequence A is labeled on the left side of the display as “ToMVMP_” and parent sequence B is labeled “UIMP_”. A ruler under the parent sequences A and B provides an indication of the location of the represented portions of the sequences and the daughter sequences are depicted row by row, (sequence by sequence) beneath the ruler. As can be determined by examination of the display, portions of some daughter sequences were inherited from parent sequence A and some portions were inherited from parent sequence B. Similarly, some portions of parent sequence B are the same as portions of parent sequence A. FIG. 7B shows a portion of a table displayed at a lower portion of FIG. 6, only on an enlarged scale. The table in FIG. 7B lists identification of each daughter sequence along with statistical data related to each of the daughter sequences.

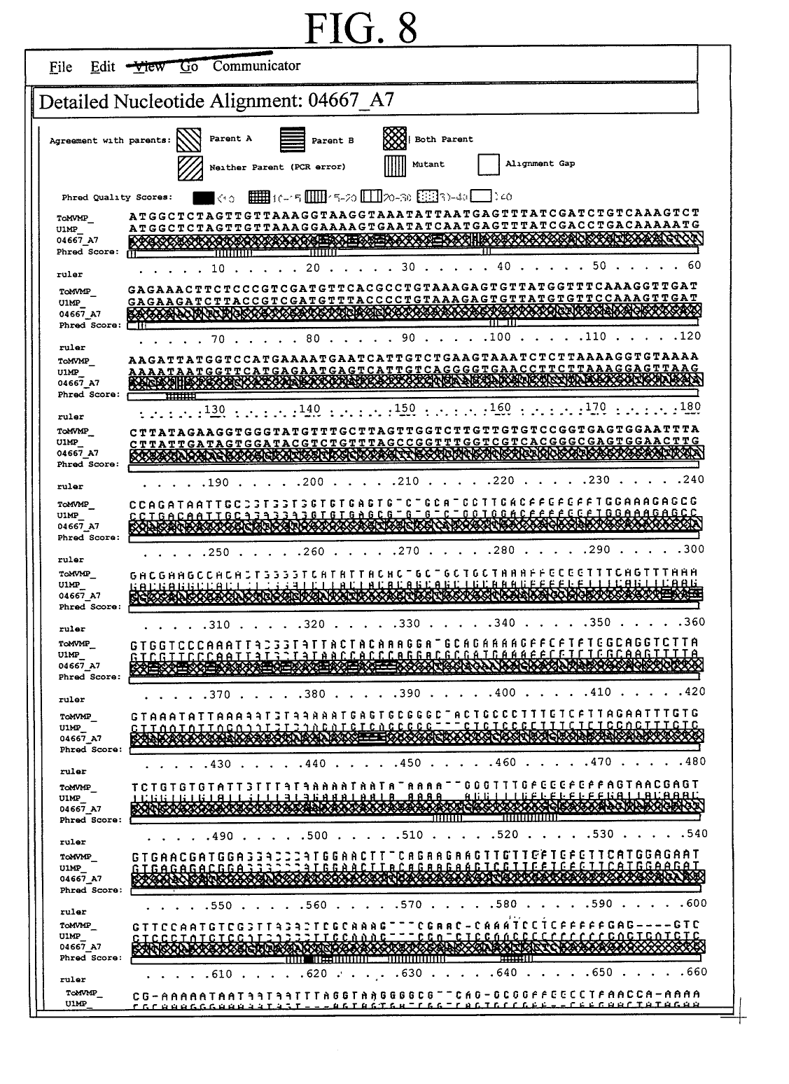

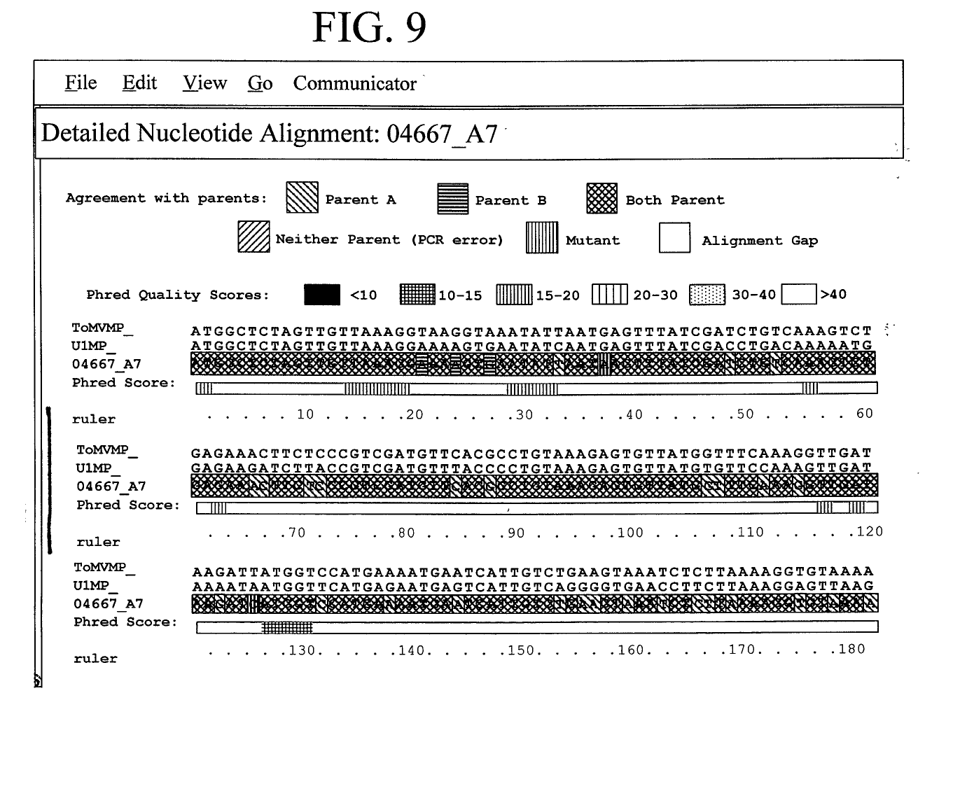

During an inspection of the sequence data represented in FIG. 6, a user may wish to look at only the parent sequences along with a single selected daughter sequence in order to provide a more careful consideration of the corresponding data. The present invention provides a link on any location on each daughter sequence depicted in FIG. 6 such that by clicking a mouse button with the mouse pointer over the selected daughter the user is shown a new display that shows the parent sequences A and B and the selected daughter sequence. As shown in FIG. 3, at step S10, a user may select a daughter sequence for closer inspection. At step S11 a detailed alignment of the parent sequences A and B and the selected daughter is generated and displayed on the user interface. An example of such a new display is shown in FIG. 8 where the actual sequence listing is depicted, letter by letter, for the parent sequence A, the parent sequence B and the selected daughter sequence. Further, each depicted nucleotide base sequence letter is highlighted with the previously mentioned colors indicating the origin of that base. In other words, the color scheme selected in the display of FIG. 4 (and FIG. 5) and displayed in FIG. 6 (and FIGS. 7A and 7B) is carried over into the display in FIG. 8 so that a user can determine from the color scheme that one colored sequence segment in the depicted daughter was inherited from parent sequence A, another colored sequence segment in the depicted daughter was inherited from parent sequence B, and so on with respect to mutant sequences, inheritance from both parent sequences and alignment gaps. A portion of FIG. 8 is shown on an enlarged scale in FIG. 9 in order to provide a clearer depiction of the detailed information provided in the display in FIG. 8. Further, a calculated Phred score is also indicated in FIGS. 8 and 9.

It should be apparent that the display features of the present invention provide a useful means for displaying processed data in a manner that allows a user to make rapid observations with respect to the displayed data.

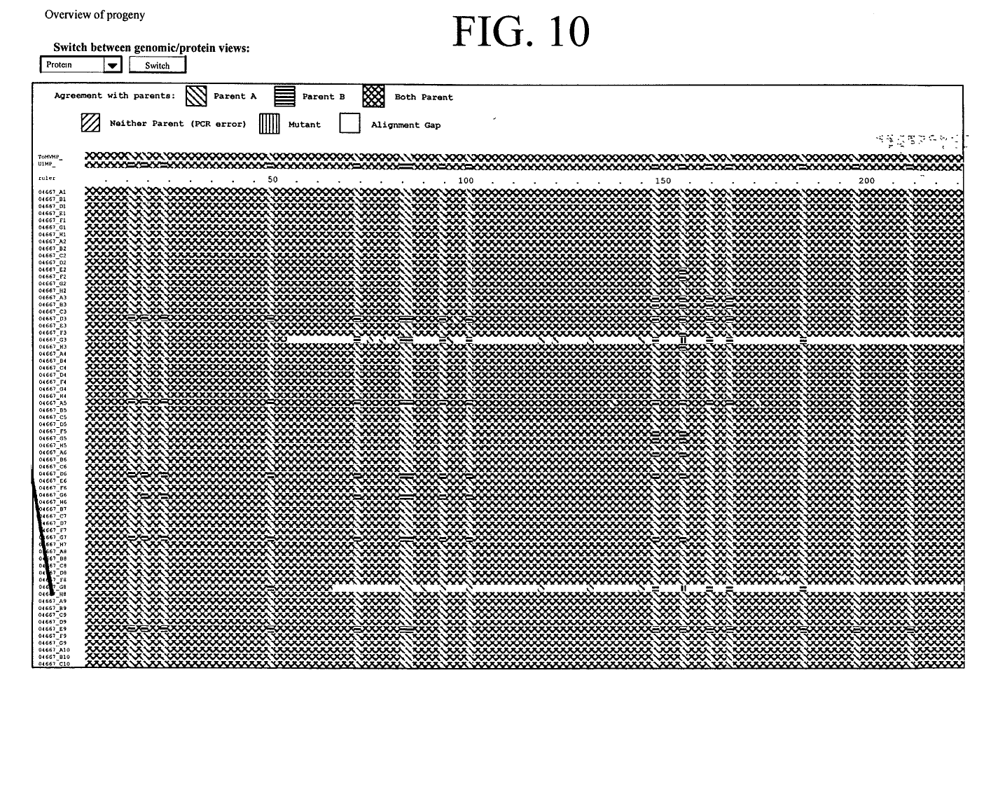

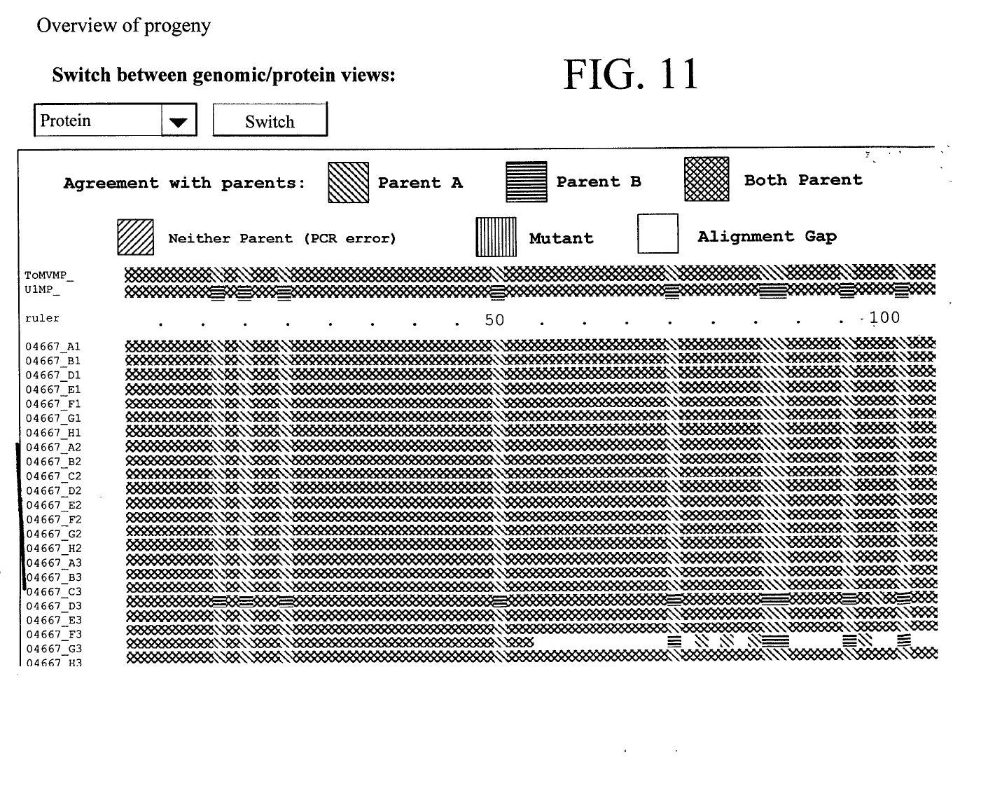

Returning to FIG. 3, at step S8, if the protein display option has have been selected, at step S13 appropriate data calculated in steps S5 and S6 is retrieved in order to provide a display of sequence data that has been translated into protein information, as depicted in FIG. 10. In a manner similar to the depiction in FIG. 6, a captured display of the protein sequence information data is shown in FIG. 10 and is also shown on an enlarged scale in FIG. 11. At the top of FIGS. 10 and 11, the selected color scheme appears. Beneath the color scheme the parent sequences A and B are represented to indicate similarities and differences between the two sequences. Next, a plurality of daughter sequences is represented using the selected color scheme to show the inheritance or legacy of the various portions thereof.

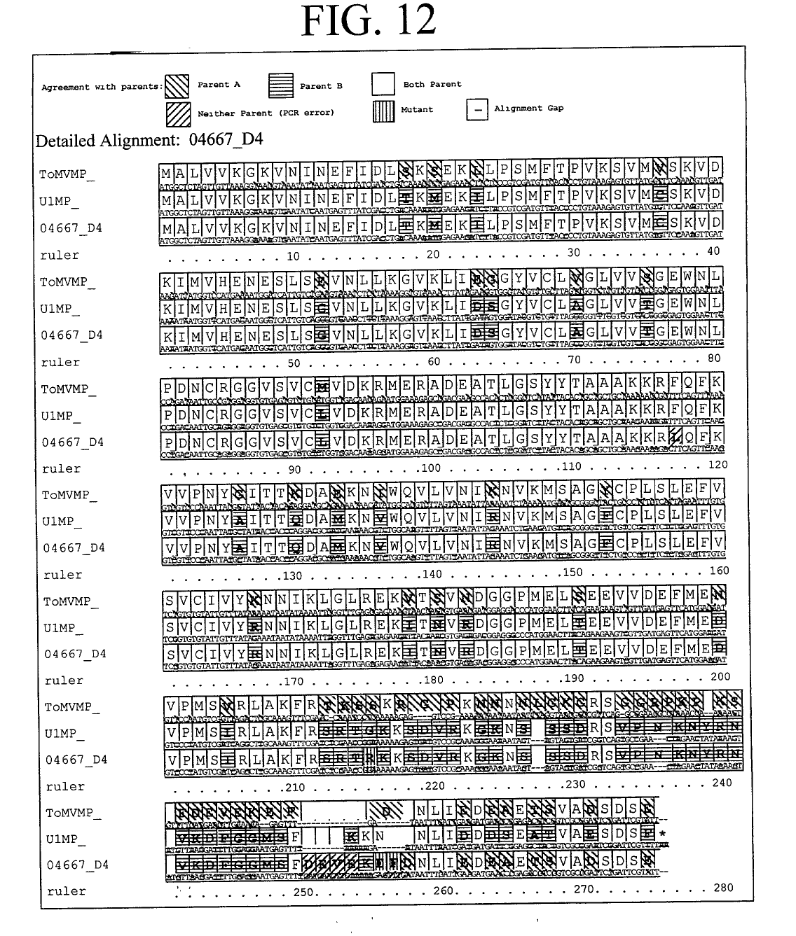

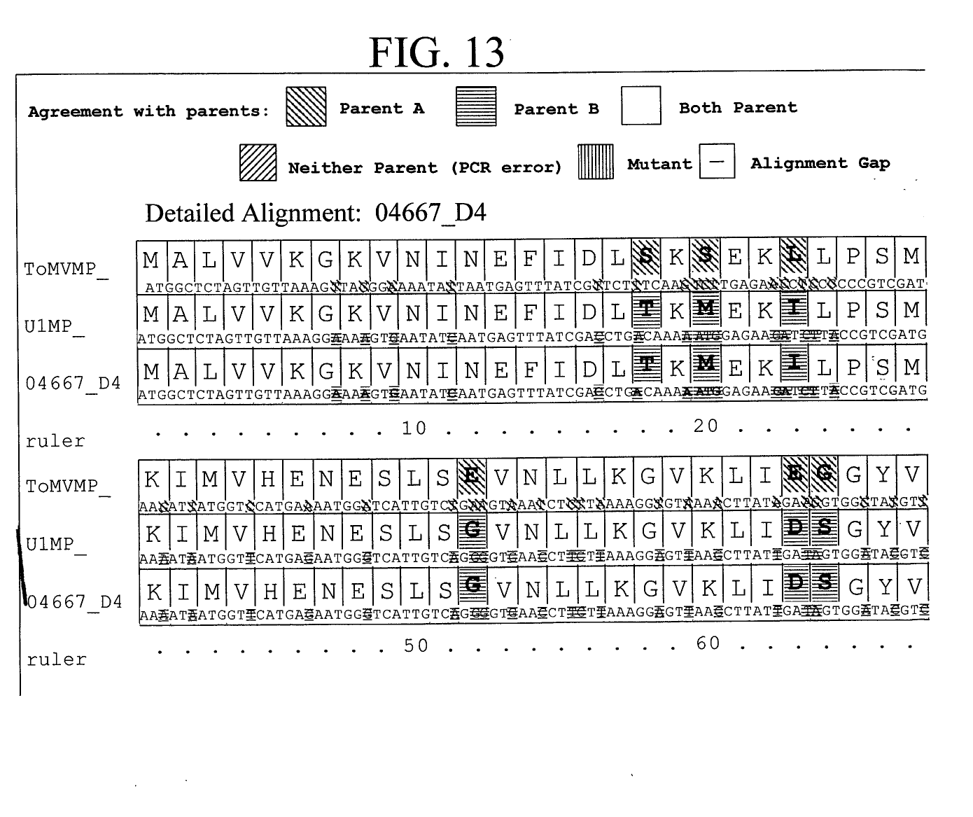

A user may point to any of the daughter sequences and click with a mouse (or digitizer) in order to generate yet another display shown in FIG. 12 where the protein sequences of parent sequence A and B and the selected daughter are depicted with the amino acid letter representation shown with a background color based upon the selected color scheme. FIG. 13 is an enlarged view of a portion of FIG. 12 to show the level of detail in the captured display image.

Returning to FIG. 3, at step S13 the captured displays in FIGS. 10 and 11 are generated and displayed. At step S14 in FIG. 3, the protein translation of a specific daughter sequence is selected and the captured display depicted in FIGS. 12 and 13 is generated. For example, at step S12, operation may return to step S9 or other predetermined step, and at step S16 operation may return to step S13, or other predetermined step.

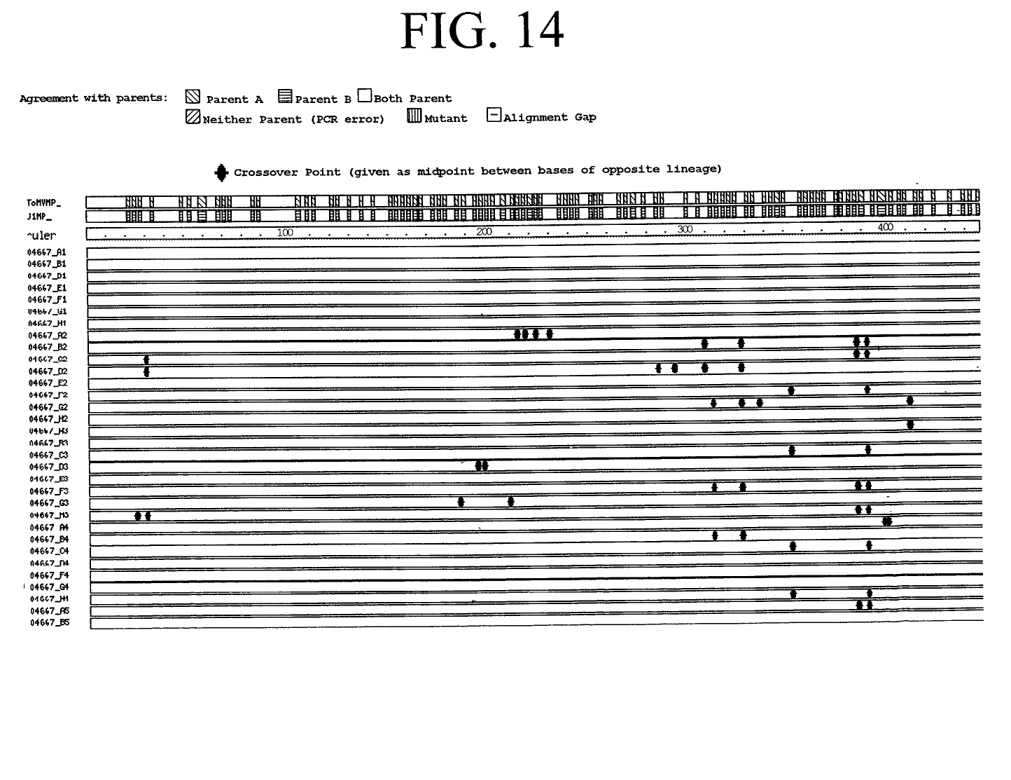

Returning to FIG. 7A, which is generated at step S9 in the flowchart shown in FIG. 3, there is a Crossover View Button displayed that, when selected by click of a mouse or other similar digitizer, links to generation of a new window which provides the screen display shown in FIG. 14. The display in FIG. 14 again lists each of the daughter sequences beneath the parent sequences, but in this display, a crossover point is indicated by a dark star. The crossover point is a determined location that signifies homology changing from one parent to another parent. In other words, at the left side of each star, the daughter sequence has homology with one patent, and to the right side of that same star, the daughter sequence has homology with the other parent. Further, the portion of the daughter sequence between any two adjacent stars has homology with one parent, and those portions on the other side of the two adjacent stars has homology with the other parent. The location of the stars in each daughter represented in the display in FIG. 14 may be arbitrary because the parent sequences have may homologous portions, and the daughters likewise typically share that homology. Therefore the display in FIG. 14 is not intended to show the origin of an entire homologous portion, but rather the stars in FIG. 14 are meant to indicate a crossover from homology with one parent to homology with the other parent. One purpose of the display in FIG. 14 is to provide a researcher with a simple way to identify homologous portions of daughter sequences. The researcher can easily return to the display in FIGS. 6 and 7A to look at a more detailed rendering of the homology, but in FIG. 14 can get a rapid sense of the degree of shuffling for each of the daughters. Specifically, the researcher can rapidly observe those daughters that show a high occurrence of inheritance crossovers.

From the display in FIG. 14, the researcher may select (click of a mouse or digitizer) one of the daughters to generate a display corresponding to the display in FIG. 8, and corresponding to steps S10 and S11 in FIG. 3.

In FIG. 3 at either step S12 or S16 the operation may return to any of a variety of predetermined points in order to repeat the analysis with a new selected daughter or to input a new set of parent and daughter sequences for analysis.

The present invention provides for a simple and easy to use graphical interface for generating visual interpretations of sequence data by aligning a plurality of sequences, then displaying the sequences side by side with visual representations of the lineage of the sequences. Lineage refers to inheritance of sub-sequences from parent sequences.

The present invention also provides a researcher with the ability to view sequences from databases or clinical studies which differ in predictable ways, such as variations in SNPs, or sequences that are manipulated or changed by any of a variety of techniques, including SNPs (single nucleotide polymorphism), chemical or physical mutagenesis, mutagenic PCR, oligonucleotide-directed mutagenesis, or growth in a DNA repair deficient microorganism (mutator strain).

It should be understood that the data manipulation and graphical interface of the present invention may also be used for comparing any sequences and need not be limited to evaluation of parent/daughter related sequences. Specifically, any group of related or similar sequences may be evaluated and compared for display using the present invention.

What is claimed is:

1. A graphical interface for displaying at least one of nucleic acid and amino acid sequence data, the sequence data representing a first parent sequence, a second parent sequence and at least one daughter sequence, where the daughter sequence includes sequence segments inherited from at least one of the first parent sequence and the second parent sequence, the graphical interface comprising:
a means for accessing sequence data of a plurality of sequences;
a means for processing sequence data, including comparing and aligning portions of the plurality of sequences with one another; and
a means for displaying the processed sequence data and the plurality of sequences for evaluation of the plurality of sequences.

2. A graphical interface as set forth in claim 1, wherein said means for displaying displays segments of the daughter sequence includes indicia indicating inheritance from at least one of the first parent sequence and the second parent sequence.

3. A graphical interface as set forth in claim 1, wherein said means for displaying displays a Phred quality score which includes indicia corresponding to segments of a daughter sequence and indicating the Phred quality score for each corresponding segment.

4. A graphical interface as set forth in claim 1, wherein said means for displaying displays segments of the daughter sequence includes indicia indicating mutant segments thereof.

5. A graphical interface as set forth in claim 1, wherein said means for displaying displays segments of the daughter sequence includes indicia indicating inheritance from the first parent sequence and the second parent sequence.

6. A graphical interface as set forth in claim 1, wherein said means for displaying displays segments of the daughter sequence includes indicia indicating inheritance from neither of the first parent sequence and the second parent sequence.

7. A graphical interface as set forth in claim 1, wherein:
said means for processing sequence data includes converting the sequence data to protein sequence data; and
in response to selection of protein sequence display, said means for displaying displays the protein sequence data.

8. A method for processing and displaying at least one of nucleic acid and amino acid sequence data, the sequence data representing a first parent sequence, a second parent sequence and at least one daughter sequence, where the daughter sequence includes sequence segments inherited from at least one of the first parent sequence and the second parent sequence, the method comprising the steps of:
accessing data relating to a plurality of sequences;
aligning the sequences;
determining inheritance of portions of the sequences; and
graphically displaying at least a portion of the plurality of sequences and inheritance characteristics of the plurality of sequences.

9. A method as set forth in claim 8, wherein said displaying step includes displaying segments of the daughter sequence in indicia indicating inheritance from at least one of the first parent sequence and the second parent sequence.

10. A method as set forth in claim 8, wherein in said displaying step a Phred quality score is displayed in indicia corresponding to portions of a daughter sequence indicating the Phred quality score for corresponding segments of the sequence.

11. A method as set forth in claim 8, wherein said displaying step includes displaying segments of the daughter sequence with indicia indicating mutant segments thereof.

12. A method as set forth in claim 8, wherein in said displaying step includes displaying segments of the daughter sequence by indicia indicating inheritance from both the first parent sequence and the second parent sequence.

13. A method as set forth in claim 8, wherein said displaying step includes displaying segments of the daughter sequence by indicia indicating inheritance from neither of the first parent sequence and the second parent sequence.

14. A method as set forth in claim 8, further comprises a processing step where sequence data is converted to protein sequence data.

15. A method as set forth in claim 14, wherein said displaying step includes displaying the daughter and parent sequences by indicia indicating inheritance from neither of the first parent sequence and the second parent sequence.

16. A graphical interface for displaying at least one of nucleic acid sequence data and amino acid sequence data, comprising:
a means for accessing sequence data representing a plurality of sequences;
a means for processing sequence data to produce processed sequence data, including comparing and aligning portions of the plurality of sequence data with one another; and
a means for displaying the processed sequence data and the plurality of sequence data for evaluation of the plurality of sequences.

17. A graphical interface as set forth in claim 16, wherein the plurality of sequences comprises:
a first parent sequence;
a second parent sequence; and
at least one daughter sequence, where the daughter sequence includes sequence segments inherited from at least one of the first parent sequence and the second parent sequence.

18. A graphical interface as set forth in claim 17, wherein said means for displaying displays segments of the daughter sequence includes indicia indicating inheritance from at least one of the first parent sequence and the second parent sequence.

19. A graphical interface as set forth in claim 17, wherein said means for displaying displays a Phred quality score by indicia corresponding to segments of a daughter sequence and indicating the Phred quality score for each corresponding segment.

20. A graphical interface as set forth in claim 17, wherein said means for displaying includes displaying segments of the daughter sequence with indicia indicating mutant segments thereof.

21. A graphical interface as set forth in claim 17, wherein said means for displaying includes displaying segments of the daughter sequence with indicia indicating inheritance from both the first parent sequence and the second parent sequence.

22. A graphical interface as set forth in claim 17, wherein said means for displaying includes displaying segments of the daughter sequence with indicia indicating inheritance from neither of the first parent sequence and the second parent sequence.

23. A graphical interface as set forth in claim 17, wherein:
said means for processing sequence data includes converting the sequence data to protein sequence data; and
in response to selection of protein sequence display, said means for displaying displays the protein sequence data.

24. A method for processing and displaying at least one of nucleic acid sequence data and amino acid sequence data, the method comprising the steps of:
accessing data corresponding to a plurality of sequences;
aligning the sequences;
determining inheritance of portions of the sequences; and
graphically displaying at least a portion of the plurality of sequences and inheritance characteristics of the plurality of sequences.

25. A method as set forth in claim 24, wherein the plurality of sequences comprises:
a first parent sequence;
a second parent sequence; and
at least one daughter sequence, where the daughter sequence includes sequence segments inherited from at least one of the first parent sequence and the second parent sequence.

26. A method as set forth in claim 25, wherein said displaying step includes displaying segments of the daughter sequence with indicia indicating inheritance from at least one of the first parent sequence and the second parent sequence.

27. A method as set forth in claim 25, wherein said displaying step includes displaying a Phred quality score with indicia corresponding to segments of a daughter sequence and indicating the Phred quality score for each corresponding segment.

28. A method as set forth in claim 25, wherein said displaying step includes displaying segments of the daughter sequence with indicia indicating mutant segments thereof.

29. A method as set forth in claim 25, wherein said displaying step includes displaying segments of the daughter sequence with indicia indicating inheritance from both the first parent sequence and the second parent sequence.

30. A method as set forth in claim 25, wherein said displaying step includes displaying segments of the daughter sequence with indicia indicating inheritance from neither of the first parent sequence and the second parent sequence.

31. A method as set forth in claim 25, further comprises a processing step where sequence data is converted to protein sequence data.
